(19) United States
(12) Patent Application Publication
(10) Pub. No.: US 2017/0020393 A1
(43) Pub. Date: Jan. 26, 2017

(54) Endoscope Having Depth Determination

(71) Applicant: Siemens Aktiengesellschaft, Muenchen (DE)

(72) Inventors: Peter Rentschler, Neuhengstett (DE); Anton Schick, Velden (DE)

(73) Assignee: Siemens Aktiengesellschaft, Muenchen (DE)

(21) Appl. No.: 15/124,169

(22) PCT Filed: Feb. 26, 2015
(86) PCT No.: PCT/EP2015/054040

§ 371 Date: Sep. 7, 2016

(30) Foreign Application Priority Data
Mar. 7, 2014 (DE) 10 2014 204 243.7

(51) Int. Cl. A61B 5/00; A61B 1/06; A61B 1/07; G01B 11/22; A61B 1/04; A61B 1/018; G02B 23/24; G02B 27/42; A61B 1/00; A61B 5/107

(52) U.S. Cl. CPC A61B 5/0084; A61B 1/00096; A61B 1/063; A61B 1/07; A61B 5/1076; A61B 5/1079; A61B 1/04; A61B 1/018; A61B 1/00009; G02B 23/2461; G02B 27/425; G02B 23/2469; G01B 11/22

(57) ABSTRACT

An endoscope for determining the depth of a partial area of a cavity by a triangulation analysis may include a projection channel for projecting a pattern onto a surface of the cavity and an imaging channel provided for imaging an image of the projected pattern reflected by the surface of the cavity. The projection channel may have at least one diffractive optical element for producing the pattern, a collimator, and a focusing lens. The focusing lens may be arranged between the collimator and the diffractive optical element.

### CROSS-REFERENCE TO RELATED APPLICATIONS

This application is a U.S. National Stage Application of International Application No. PCT/EP2015/054040 filed Feb. 26, 2015, which designates the United States of America, and claims priority to DE Application No. 10 2014 204 243.7 filed Mar. 7, 2014, the contents of which are hereby incorporated by reference in their entirety.

### TECHNICAL FIELD

The invention relates to an endoscope for determining the depth of a portion of a cavity.

### BACKGROUND

The number of minimally invasive operations has increased steadily in recent years. Here, endoscopes (3D endoscopes) which enable a depth determination of a cavity in a patient to be examined and, at the same time, enable an imaging method are playing an ever greater role. According to the prior art, a plurality of accesses (ports) are established for determining the depth, for example of an abdominal cavity of the patient. However, in minimally invasive surgery, attempts are being made to minimize the number of ports.

In order to achieve a minimum of ports, attempts are being made to complement endoscopes known according to the prior art in such a way that a depth determination is made possible per se. A substantial disadvantage of such 3D endoscopes is that only little installation space is available for the setup and integration of optics for determining the depth. As result of this, this adversely affects, in particular, the resolution, the image quality, the depth of field and the field of vision of the optics, as a result of which, overall, the optical performance of the depth determination of known 3D endoscopes is reduced.

3D endoscopes known from the prior art typically use the stereoscopy principle for determining the depth. To this end, two imaging channels, which each comprise imaging optics, are guided through the 3D endoscope. From the images of the cavity, which are acquired from different points of view by means of the two imaging channels, it is possible to determine the depth of the cavity from the difference between pixels of the images. A basic problem in stereoscopy is the correspondence problem. The depth determination emerges from a pixel in the first image, which was imaged by means of one imaging channel, and the modified position of the pixel in the second image, which was imaged by means of the other imaging channel. Here, the pixel in the first image and the pixel in the second image must be identifiable as the same pixel. If this identifiability is not present, there is a correspondence problem.

In the case of surfaces with little texture, e.g. blood, or in the case of organic tissue, there typically only are a small number of pixels available, and so the correspondence problem is exacerbated when using stereoscopy in minimally invasive surgery.

For the purposes of solving the correspondence problem, the prior art proposes so-called active triangulation methods, wherein one imaging channel in the 3D endoscope is replaced by a projection channel during active triangulation. Although this largely solves the correspondence problem, the optical performance of the imaging optics of the 3D endoscope is disadvantageously reduced. Such a reduction is not admissible, particularly in minimally invasive surgery.

Color-coded triangulation methods for determining the depth were also found to be problematic since the organic tissues are typically surrounded by blood, and so there is almost complete absorption of blue and green portions of the projected color pattern. As a result, imperfections arise in the image of the color pattern, which in turn lead to a correspondence problem.

### SUMMARY

One embodiment provides an endoscope for determining the depth of a portion of a cavity, comprising at least one projection channel for projecting a pattern onto a surface of the cavity and at least one imaging channel provided for imaging an image of the projected pattern reflected by the surface of the cavity, wherein the projection channel comprises at least one diffractive optical element for generating the pattern, a collimator, and a focusing lens, wherein the focusing lens is arranged between the collimator and the diffractive optical element.

In one embodiment, the diffractive optical element, the collimator, and the focusing lens are arranged in a portion of the projection channel, wherein the portion has an axial extent of at most 5 mm with respect to an optical axis.

In one embodiment, the focusing lens, and the diffractive optical element are arranged in the projection channel in coaxial fashion with respect to the optical axis.

In one embodiment, a cross-sectional area of the imaging channel is greater than a cross-sectional area of the projection channel.

In one embodiment, the cross-sectional area of the projection channel is less than or equal to 2 mm².

In one embodiment, the cross-sectional area of the imaging channel is greater than or equal to 2 mm².

In one embodiment, the endoscope includes a projection channel which is optically coupled to a single-mode fiber.

In one embodiment, the single-mode fiber is optically coupled to a laser.

In one embodiment, the imaging channel is optically coupled to a camera for recording the image of the reflected pattern.

In one embodiment, the camera is a three-chip camera.

In one embodiment, the endoscope includes an instrumentation channel.

Another embodiment, provides a method for determining the depth of a portion of a cavity, in which an endoscope with a projection channel comprising a diffractive optical element, a collimator, and a focusing lens arranged between the collimator and the diffractive optical element and with an imaging channel is used, wherein a pattern is projected onto a surface of the cavity by means of the projection channel and an image of the pattern reflected by the surface is imaged by means of the imaging channel, wherein the pattern is generated by means of the diffractive optical element.

In one embodiment, a point pattern is generated by means of the diffractive optical element.

In one embodiment, the depth determination of the portion of the cavity is carried out by means of the distances between points of the point pattern.

### BRIEF DESCRIPTION OF THE DRAWINGS

Example aspects and embodiments of the invention are described below with reference to the drawings, in which:

FIG. 1 shows a section of a projection channel of an endoscope, wherein the projection channel comprises a diffractive optical element;

FIG. 2 shows a sectional illustration of an endoscope with a projection channel and an imaging channel; and

FIG. 3 shows a further sectional illustration of an endoscope with a projection channel and an imaging channel.

### DETAILED DESCRIPTION

Embodiments of the present invention provide an endoscope with improved optical depth determination, and a corresponding method.

Some embodiments provide an endoscope for determining the depth of a portion of a cavity, which endoscope includes at least one projection channel for projecting a pattern onto a surface of the cavity and at least one imaging channel provided for optically imaging an image of the projected pattern reflected by the surface of the cavity, wherein the projection channel comprises at least one diffractive optical element for generating the pattern.

In some embodiments, the pattern enabling the depth determination of the portion of the cavity is generated by means of the diffractive optical element. Diffractive optical elements (abbreviated DOEs) are optical elements which are embodied for spatially structuring light, wherein the structuring is carried out by means of diffraction. By way of example, an optical grating is a diffractive optical element. A pattern, in particular a point pattern, is generated by means of the diffractive optical element, said pattern enabling a depth determination of the portion of the cavity after an evaluation.

By arranging the diffractive optical element in the projection channel of the endoscope, it is advantageously possible to form a DOE projector, for example by means of further optical components. Here, a DOE projector is a projector which comprises a diffractive optical element instead of a slide. It is particularly advantageous that the required installation space of such a DOE projector is lower—compared to projectors with slides.

Advantageously, a distance which is as large as possible is enabled between the projection channel and the imaging channel as a result of the low installation space requirements of the DOE projector. This is advantageous because the distance corresponds to a triangulation base of the triangulation, with the enlarged triangulation base, in particular, leading to an improved depth resolution of the endoscope.

Some embodiments provide a method for determining the depth of a portion of a cavity, in which an endoscope with a projection channel comprising a diffractive optical element, a collimator, and a focusing lens arranged between the collimator and the diffractive optical element and with an imaging channel is used, wherein a pattern is projected onto a surface of the cavity by means of the projection channel and an image of the pattern reflected by the surface is imaged by means of the imaging channel, wherein the pattern is generated by means of the diffractive optical element.

In some embodiments, a pattern generated by means of the diffractive optical element is projected onto the surface of the portion of the cavity and an image of the pattern reflected by the surface is imaged by means of the imaging channel. Advantages which are similar and of equal value to the aforementioned endoscope according to the invention emerge.

In some embodiments, the projection channel comprises a collimator and a focusing lens, wherein the focusing lens is arranged between the collimator and the diffractive optical element.

Light introduced into the projection channel is collimated by means of a lens. The projection channel may comprise a further lens which focuses the light introduced into the projection channel onto a working distance of the endoscope. In other words, the lens mentioned first forms a collimator, a plurality of lenses form collimator optics, and the lens mentioned second forms the focusing lens. Here, provision is made of a diffractive optical element which also takes into account the focusing of the light when generating the pattern.

In some embodiments, the diffractive optical element, the collimator, and the focusing lens are arranged in a portion of the projection channel, said portion having an axial extent of at most 5 mm with respect to an optical axis.

Here, the optical axis in the portion advantageously extends coaxially with an axis of symmetry of the projection channel.

Provision is made for the diffractive optical element, the collimator, and the focusing lens to be arranged coaxially with respect to the optical axis in the projection channel. A DOE projector arranged in the projection channel of the endoscope is formed by arranging the diffractive optical element, the collimator, and the focusing lens in the portion which has an axial extent of at most 5 mm. Advantageously, this DOE projector has low installation space requirements such that the DOE projector can be installed in endoscopes known from the prior art. To this end, a collimator with a diameter of at most 1 mm is preferred. As a result of the aforementioned small diameter of the collimator, it is advantageously possible to enlarge the triangulation base such that the depth resolution of the endoscope is improved.

In one embodiment, a cross-sectional area of the imaging channel is greater than a cross-sectional area of the projection channel.

Here, the area emerging from a section through the imaging channel or the projection channel perpendicular to the optical axis of the respective channel is referred to as cross-sectional area in each case.

In some embodiments, the cross-sectional area of the projection channel which is reduced in relation to the imaging channel is sufficient to arrange the DOE projector in the projection channel. As a result of the low installation space requirements of the DOE projector, installation space available in the endoscope is saved, and so more installation space can be used for the imaging channel and, consequently, for improving the imaging optics, said imaging optics being arranged in the imaging channel.

In one embodiment, a cross-sectional area of the projection channel is less than or equal to 2 mm².

As a result, a very small projection channel is advantageously formed, and so, consequently, additional installation space can be saved in the endoscope. Here, the imaging channel preferably has a cross-sectional area of at least 2 mm². In particular, the cross-sectional area of the imaging channel lies in the range of 25 mm² to 64 mm², wherein larger imaging channels may be provided.

In one embodiment, the projection channel is optically coupled to a single-mode fiber.

As a result, light guided by means of the single-mode fiber (SMF) is introduced into the projection channel by means of the single-mode fiber. The single-mode fiber advantageously only guides one light mode, and so interferences between a plurality of light modes, which could lead to interference in the projected pattern, are avoided.

In some embodiments, the single-mode fiber is coupled to a laser, wherein the light of the laser is introduced into the projection channel by way of the single-mode fiber. Here, the wavelength of the laser can be adapted to the application in the surgery for the purposes of generating an ideal point contrast, for example in the blue spectral range. It is particularly advantageous that, for example by way of an interference filter, a bothersome influence of daylight and/or artificial light is reduced by using a laser as a light source.

In one embodiment, the imaging channel is optically coupled to a camera for recording the image of the reflected pattern.

A camera which is embodied as three-chip camera is particularly preferred. Here, the camera has a chip for the red spectral range, a chip for the green spectral range and a chip for the blue spectral range of the recorded image. Advantageously, this enables an approximately complete image of the reflected pattern imaged by the imaging channel.

In one embodiment, the endoscope comprises an instrumentation channel.

Advantageously, surgical tools required for minimally invasive surgery are inserted into the cavity through the instrumentation channel. Installation space is saved by arranging a diffractive optical element in the projection channel, said installation space in turn being able to be used for the instrumentation channel.

In one embodiment, a point pattern is generated by means of the diffractive optical element.

Here, the individual points of the point pattern correspond to the orders of diffraction of the diffractive optical element. In other words, a point pattern is generated by means of the diffractive optical element by constructive and destructive interference of the light introduced into the projection channel. The point pattern is projected onto the surface of the portion of the cavity and enables a depth determination of the portion by evaluating the distances between the points. Hence, the correspondence problem in the case of active triangulation is reduced by the point pattern which is generated by diffraction by means of the diffractive optical element.

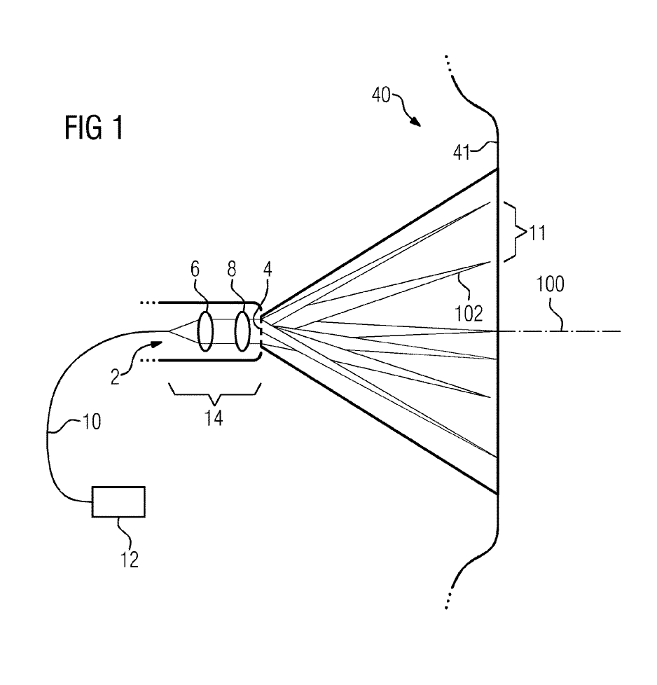

FIG. 1 shows a schematic section of a projection channel 2 of an endoscope 1 (not depicted here). Here, the projection channel 2 may comprise a diffractive optical element 4. Furthermore, a collimator 6 and a focusing lens 8 are arranged within the projection channel 2. Further optical components, e.g. lenses, mirrors, objectives and/or beam-deflection apparatuses may be provided. Moreover, provision can be made of a plurality of projection channels. It is not mandatory for the projection channel to extend through the entire endoscope 1. By way of example, each portion of the endoscope 1 which comprises at least one diffractive optical element 2 can be considered to be a projection channel.

The collimator 6, the focusing lens 8, and the diffractive optical element 4 are arranged coaxially with respect to an optical axis 100 of the projection channel 2. Here, the aforementioned elements 4, 6, 8 are arranged in a portion 14 of the projection channel 2, said portion 14 having an axial extent of almost 3 mm with respect to the optical axis 100. By forming a DOE projector in the projection channel 2 of the endoscope 1 by means of the diffractive optical element 4, it is possible to save installation space which can be used differently, for example for an instrumentation channel (not depicted here).

The projection channel 2 is optically coupled to a laser 12 or a light-emitting diode by means of a single-mode fiber 10. The light from the laser 12 is guided in the single-mode fiber 10 and introduced into the projection channel 2, collimated by means of the collimator 6, and focused by means of the focusing lens 8. After the focusing lens 8, the light from the laser 12 is guided to the diffractive optical element 4 such that a point pattern is projected onto a surface 41 of a portion of the cavity 40 by way of diffraction of the light at the diffractive optical element 4. Here, the individual points of the point pattern correspond to the orders of diffraction 102 (principal maxima and subsidiary maxima of an intensity distribution of the diffracted light).

The diffractive optical element 4 is configured in such a way that the distances 11 between the individual points of the point pattern vary, with the correspondence problem being solved or reduced by the variation of the distances 11. In other words, an assignment of the points of the reflected pattern is successful by way of a comparison with an original point pattern, said original point pattern for example being generated from a projection of the point pattern onto a plane surface (calibration). Consequently, the varying distances of a point from its neighboring points generate a code which is used to solve or improve the correspondence problem.

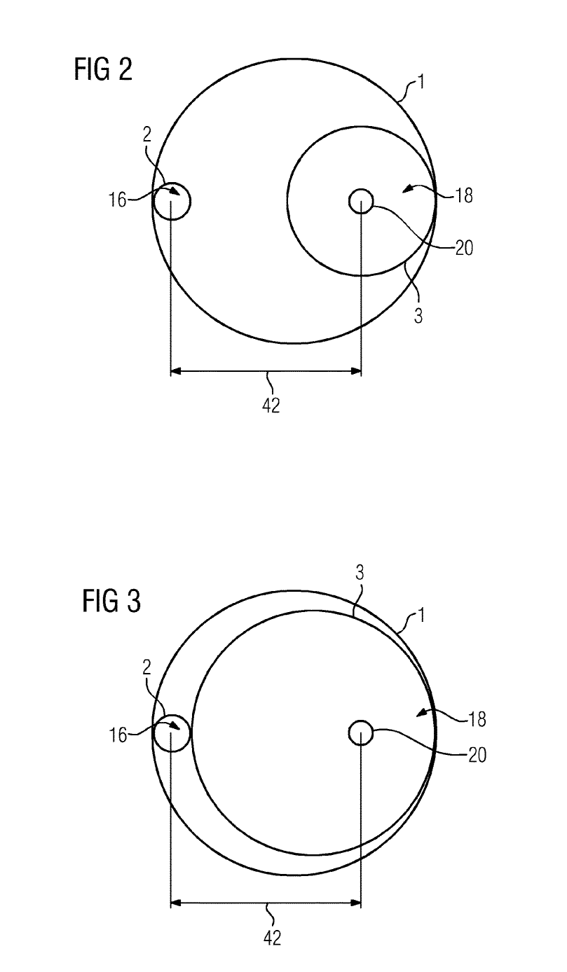

FIG. 2 shows a schematic sectional illustration of an endoscope 1, with the section extending perpendicular to an optical axis 100 of a projection channel 2. Furthermore, FIG. 2 shows an imaging channel 3, wherein provision may be made of a plurality of imaging channels 2. An endoscope 1 with two imaging channels 3 and one projection channel 2 is preferred.

By arranging or embodying a DOE projector in the projection channel 2 of the endoscope 1, it is possible for a cross-sectional area 16 of the projection channel 2 to be significantly smaller than a cross-sectional area 18 of the imaging channel 3. Consequently, the optical performance of imaging optics (not depicted here) arranged in the imaging channel 3 is substantially improved by the enlarged cross-sectional area 18 of the imaging channel 3.

Where possible, the projection channel 2 is arranged at an outer edge region of the endoscope 1. Furthermore, the imaging channel 3 is arranged at a further outer edge region of the endoscope 1, which lies opposite the projection channel 2. As a result, a triangulation base 42 between a pupil 20 of the imaging channel 3 and the projection channel 2 is advantageously enlarged, as a result of which the depth resolution of the endoscope 1 is improved. Here, the triangulation base lies in a range of 5 mm to 10 mm.

FIG. 3 depicts a further schematic sectional illustration of an endoscope 1, wherein the section extends perpendicular to an optical axis 100 of a projection channel 2 and/or of an imaging channel 3. Here, the endoscope 1 illustrated schematically in FIG. 3 has a diameter of at least 10 mm. In FIG. 3, the cross-sectional area 18 of the imaging channel 3 has an embodiment which is as large as possible such that the imaging channel 3 almost completely takes up the entire installation space of the endoscope 1. This is possible since the projection channel 2 requires a comparatively small cross-sectional area 16 as a result of the DOE projector or as a result of the diffractive optical element 4, wherein a comparison with projectors having slides should be drawn. As a result, overall, the optical depth determination and the optical performance of the endoscope 1 are further improved.

The projection channel 2 and/or the imaging channel 3 can comprise further optical components, e.g. lenses, mirrors, gratings, beam splitters and/or prisms and/or entire optical apparatuses, e.g. objectives. In particular, the imaging channel 3 can be formed by an objective. Here, a camera, for example a three-chip camera, can be arranged at the objective and/or be integrated into the objective. Here, the images are guided by way of optical fibers, in particular by means of a single-mode fiber 10.

Even though the invention was, in part, illustrated and described more closely by the preferred exemplary embodiments, the invention is not restricted by the disclosed examples and other combinations can be derived therefrom by a person skilled in the art, without departing from the scope of protection of the invention.

What is claimed is:

1. An endoscope for determining the depth of a portion of a cavity by a triangulation calculation, the endoscope comprising:
at least one projection channel that projects a pattern onto a surface of the cavity, and
at least one imaging channel that images an image of the projected pattern reflected by the surface of the cavity,
wherein the projection channel comprises:
at least one diffractive optical element that generates the pattern,
a collimator, and
a focusing lens, and
wherein the focusing lens is arranged between the collimator and the diffractive optical element.

2. The endoscope of claim 1, wherein the diffractive optical element, the collimator, and the focusing lens are arranged in a portion of the projection channel, wherein the portion has an axial extent of at most 5 mm along a direction of an optical axis.

3. The endoscope of claim 1 wherein the collimator, the focusing lens, and the diffractive optical element are arranged in the projection channel in coaxial fashion with respect to an optical axis.

4. The endoscope of claim 1, wherein a cross-sectional area of the imaging channel is greater than a cross-sectional area of the projection channel.

5. The endoscope of claim 4, wherein the cross-sectional area of the projection channel is less than or equal to 2 mm².

6. The endoscope of claim 4, wherein the cross-sectional area of the imaging channel is greater than or equal to 2 mm².

7. The endoscope of claim 1, comprising a projection channel which that is optically coupled to a single-mode fiber.

8. The endoscope of claim 7, wherein the single-mode fiber is optically coupled to a laser.

9. The endoscope of claim 1, wherein the imaging channel is optically coupled to a camera for recording the image of the reflected pattern.

10. The endoscope of claim 9, wherein the camera comprises a three-chip camera.

11. The endoscope of claim 1, comprising an instrumentation channel.

12. A method for determining a depth of a portion of a cavity, comprising:
providing an endoscope with a projection channel comprising a diffractive optical element, a collimator, and a focusing lens arranged between the collimator and the diffractive optical element, and an imaging channel,
projecting, via the projection channel, a pattern onto a surface of the cavity, wherein the pattern is generated by the diffractive optical element;
imaging, by the imaging channel, an image of the pattern reflected by the surface, and
performing a triangulation calculation based on the imaged pattern to determine the depth of the portion of the cavity.

13. The method of claim 12, comprising generating a point pattern by the diffractive optical element.

14. The method of claim 13, comprising determining the depth of the portion of the cavity based on distances between points of the point pattern.
